(19) United States
(12) Patent Application Publication
(10) Pub. No.: US 2017/0156475 A1
(43) Pub. Date: Jun. 8, 2017

(54) APPARATUS FOR CHANGING A TEMPERATURE OF A COMPOSITION OR TOOL

(71) Applicant: Lombardi Design & Mfg., Freeport, NY (US)

(72) Inventors: Carl M. Lombardi, Freeport, NY (US); Jack Albanese, Freeport, NY (US)

(21) Appl. No.: 15/231,900

(22) Filed: Aug. 9, 2016

Related U.S. Application Data
(60) Provisional application No. 62/211,522, filed on Aug. 28, 2015.

(51) Int. Cl. A45D 44/00; A61M 35/00

(52) U.S. Cl. CPC A45D 44/00; A61M 35/003; A61M 2205/3606; A61M 2205/36

(57) ABSTRACT

The specification relates to an apparatus for changing the temperature of a composition or tool that includes a top portion and a bottom portion. The top portion can include (1) a valve receiver capable of receiving a valve of a container, (2) an exchange chamber, (3) an aperture for communicatively connecting the valve receiver to the exchange chamber, and (4) an item holder. The item holder is capable of holding a removable item and can be enclosed within the exchange chamber. The bottom portion can include a base and sides with the sides extending upwards from the base. The sides can form an open cavity capable of receiving the container. The bottom portion can further be movably coupled to the top portion.

### CROSS REFERENCE TO RELATED APPLICATIONS

This application claims benefit to U.S. Provisional Application Ser. No. 62/211,522 filed on Aug. 28, 2015.

### BACKGROUND

The subject matter described herein relates to an apparatus for changing the temperature of a composition or tool.

Cosmetic and therapeutic treatments can be enhanced when a composition or tool to be applied to the skin or another body part of a user is cooled or heated to a desired temperature. This is beneficial when the skin has minor irritations or is applied on to a body part with heighted senses, e.g., applying a cooled composition or tool to a user's sunburn creates a cooling sensation and acts as a pain reliever, applying a cooled applicator to a user's face causes a relaxing sensation, and applying a heated composition to a sore muscle can further help to relax and soothe the muscle.

Some compositions also have increased efficacy when cooled or heated to certain temperatures. These compositions are sometimes stored in refrigerators or freezers or can be heated in an oven or microwave. This, however, limits the usage of such products to home or office use as the products quickly return to ambient temperature when removed from the appliance.

### SUMMARY

The disclosed technology relates to an apparatus for changing the temperature of a composition or tool. The apparatus allows a cooling or heating medium to surround and cool or heat an item in an exchange chamber and allows the cooling or heating medium to exit the exchange chamber in a controlled manner that is safe and effective.

In one implementation, an apparatus can comprise a top portion and a bottom portion. The top portion can include (1) a valve receiver, the valve receiver capable of receiving a valve of a container, (2) an exchange chamber, the exchange chamber having an open position and a closed position, (3) at least one aperture, the at least one aperture communicatively connecting the valve receiver to the exchange chamber, and (4) an item holder, the item holder being enclosed within the exchange chamber when the exchange chamber is in a closed position, the item holder capable of holding a removable item; and the bottom portion can include a base and sides, the sides extending upwards from the base, the sides forming an open cavity, the open cavity capable of receiving the container, wherein the bottom portion is movably coupled to the top portion.

In some implementations, the top portion can further include sides extending downwards, the sides of the top portion fitting within the open cavity of the bottom portion in a slidable configuration, so that, when a downward pressure is applied to the top portion, the top portion is capable of actuating the valve of the container thereby releasing a pressurized medium into the exchange chamber.

In some implementations, the top portion can further include a lid movably coupled to the top portion. In some implementations, the lid can include a portion of the exchange chamber. In some implementations, the lid can be connected to the top portion with a lever. In some implementations, the lever can include an extender that actuates a valve block, the valve block preventing actuation of the valve when the lid is in an open position.

In some implementations, the exchange chamber can include at least one opening for releasing excess gas pressure from within the exchange chamber.

In some implementations, the removable item can be a wand having a handle and a metallic head. In some implementations, the item holder can include a seat for holding the metallic head and the handle of the wand.

In some implementations, the removable item can be a tray for holding a composition. In some implementations, the item holder can include a seat for holding the tray.

In some implementations, the top portion can further include a shell, the shell being fixably coupled to the top portion, the shell forming a portion of the exchange chamber. In some implementations, the item holder can pivot into a space created between the top portion and the shell.

In some implementations, the container can be a gas container or an electric device. In some implementations, the apparatus can include an outer casing and a twist-on cap.

In another implementation, an apparatus can comprise: a top portion and a bottom portion. The top portion can include (1) a valve receiver, the valve receiver receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder further holding a composition packet; and a bottom portion, the bottom portion can include a base and sides, the sides extending upwards from the base, the sides forming an open cavity, the open cavity receiving the container, the bottom portion is movably coupled to the top portion.

In some implementations, the apparatus can include a lid; and a lever connected to the lid, the lever including an extender that actuates a valve block, the valve block preventing actuation of the valve when the lid is in an open position. In some implementations, the exchange chamber includes at least one opening for releasing excess gas pressure from within the exchange chamber.

In another implementation, an apparatus can comprise: a top portion and a bottom portion. The top portion can include (1) a valve receiver, the valve receiver receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder holding a wand having a handle and metallic head. The bottom portion cam include a base and sides, the sides extending upwards from the base, the sides forming an open cavity, the open cavity receiving the container, the bottom portion is movably coupled to the top portion.

In some implementations, the exchange chamber can include at least one opening for releasing excess gas pressure from within the exchange chamber.

In another implementation, an apparatus can comprise: a top portion, the top portion includes (1) a valve receiver, the valve receiver capable of receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder capable of holding a removable item; and a bottom portion, the bottom portion includes a base and an actuator, wherein the actuator is movably coupled to the base and is capable of actuating the valve of the container thereby releasing a pressurized medium into the exchange chamber.

In some implementations, the top portion and bottom portion can be secured to each other with attachment threads.

In some implementations, the apparatus can further comprise: a recess, the recess allowing the item holder to pivot from an open position to a closed position within and out of the recess.

In some implementations, the actuator can further include a seat capable of receiving the container. In some implementations, the seat can be positioned slightly above a bottom wall of the actuator so as to create a space beneath the container wherein the space creates an insulating distance between a bottom of the container and the bottom wall of the actuator.

One advantage of the disclosed technology is that the apparatus allows a user to easily insert and remove a one-time-use cosmetic packet or reusable applicator into an exchange chamber of the apparatus thereby achieving a more effective heating/cooling of the packet/applicator in safe manner.

Another advantage of the disclosed technology is that the apparatus creates a pressure relief system allowing the dispensed gas to be controlled so that excess gas is not forced from any seams in the device causing injuries to a user.

### BRIEF DESCRIPTION OF THE DRAWINGS

FIG. 1 is a perspective view of a first implementation of the disclosed technology;

FIG. 2 is a sectional view of the first implementation of the disclosed technology;

FIG. 3 is a front view of the first implementation of the disclosed technology;

FIG. 4 is a side view of the first implementation of the disclosed technology;

FIG. 5 is a top view of the first implementation of the disclosed technology;

FIG. 6 is a perspective view of a second implementation of the disclosed technology;

FIG. 7 is a sectional view of the second implementation of the disclosed technology;

FIG. 8 is a front view of the second implementation of the disclosed technology;

FIG. 9 is a side view of the second implementation of the disclosed technology;

FIG. 10 is a top view of the second implementation of the disclosed technology;

FIG. 11 is a perspective view of a third implementation of the disclosed technology;

FIG. 12 is a sectional view of the third implementation of the disclosed technology;

FIG. 13 is a front view of the third implementation of the disclosed technology;

FIG. 14 is a sectional view of a fourth implementation of the disclosed technology;

FIG. 15 is a front view of the fourth implementation of the disclosed technology;

FIG. 16 is a side view of the fourth implementation of the disclosed technology;

FIG. 17 is a perspective view of the fourth implementation of the disclosed technology; and

FIG. 18 is a top view of the fourth implementation of the disclosed technology;

FIG. 19 is a perspective view of a fifth implementation of the disclosed technology;

FIG. 20 is a front view of the fifth implementation of the disclosed technology;

FIG. 21 is a sectional view of the fifth implementation of the disclosed technology;

FIG. 22 is a perspective view of a sixth implementation of the disclosed technology;

FIG. 23 is a front view of the sixth implementation of the disclosed technology;

FIG. 24 is a sectional view of the sixth implementation of the disclosed technology;

FIG. 25 is a perspective view of a seventh implementation of the disclosed technology;

FIG. 26 is a front view of the seventh implementation of the disclosed technology;

FIG. 27 is a sectional view of the seventh implementation of the disclosed technology;

FIG. 28 is a perspective view of an eighth implementation of the disclosed technology;

FIG. 29 is a front view of the eighth implementation of the disclosed technology;

FIG. 30 is a sectional view of the eighth implementation of the disclosed technology; and

FIGS. 31-40 are additional views of the eighth implementation of the disclosed technology.

### DETAILED DESCRIPTION

The specification relates to a device for changing a temperature of a packet or an applicator used for medicinal or cosmetic purposes, e.g., skin or muscle treatments.

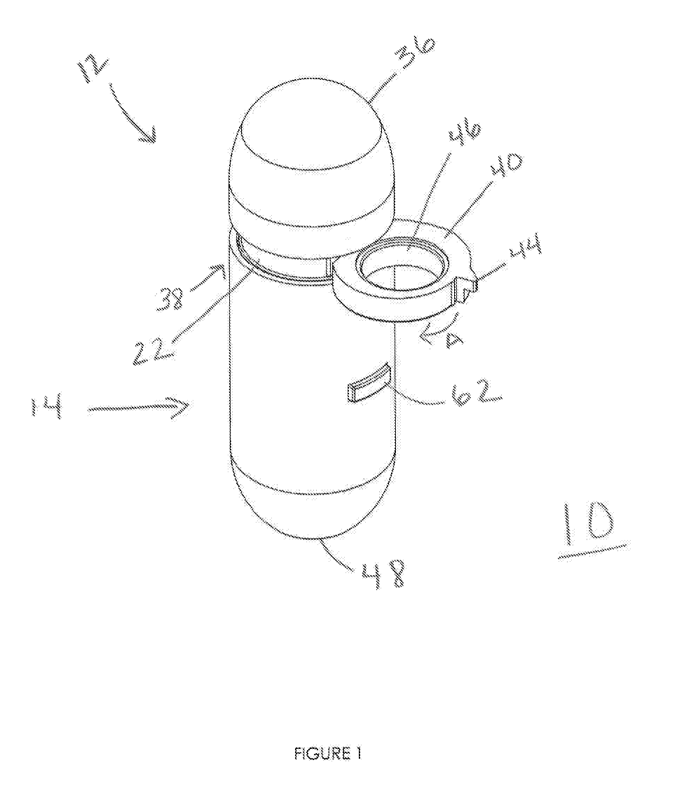

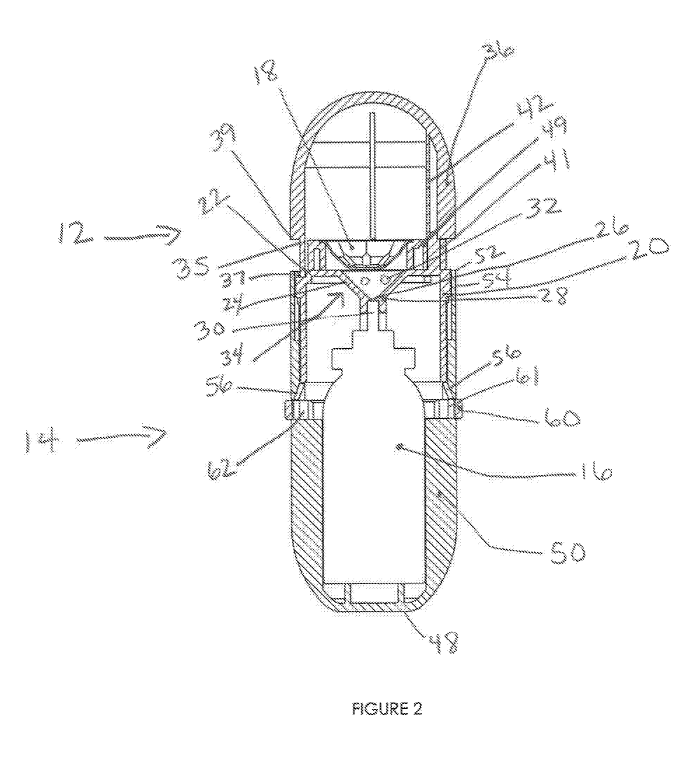

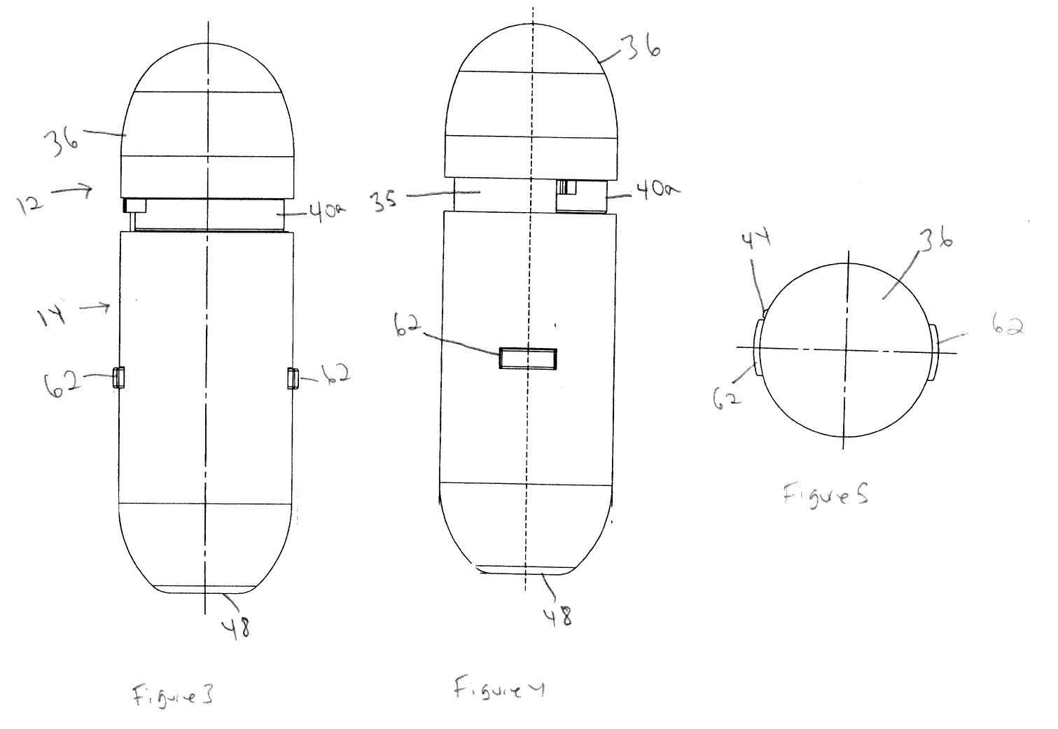

FIGS. 1-5 show an implementation of the disclosed technology. The disclosed technology can be a device 10 that includes a top portion 12 for receiving a pressurized substance from a valve-operated container 16. The container 16 can contain a coolant stored as a liquid gas or the container can be a battery-operated or a plugged-in appliance capable of producing pressurized cold or hot air. If the container is a cooling container, it can be an aerosol container containing a refrigerant. This refrigerant can be, e.g., a hydrocarbon, i.e., 2,3-butane, dimethyl ether, propane or a Freon, i.e., HFC-134a. If the container is a heating container, the container can be a battery-operated electric heating device that allows a heating element and fan to produce a pressurized heated gas.

The device 10 can also include a bottom portion 14 for retaining and supporting the container 16. The top portion 12 and bottom portion 14 can be slidably fitted together so to actuate a valve 30 of the container 16 for delivery of the pressurized substance into an exchange chamber 34 located in the top portion 12 thereby cooling or heating an item 18, e.g., a composition packet or an applicator.

The top portion 12 can have a main section 20 that is cylindrical in shape but other shapes and configurations are contemplated. The main section 20 includes a top surface 22. The top surface 22 can be circular, substantially flat and include an indentation 24 at its center. The indentation 24 can be cone-shaped, however, other configurations are contemplated.

The indentation can form a portion of an exchange chamber 34, as will be described more fully below. In the bottom-center of the indentation 24, the indentation can include an aperture 26 that communicatively connects to a valve receiver 28. The valve receiver 28 can protrude downward from a center point of the indentation 24 and be, for example, tube shaped. The inner portion of the valve receiver 28 can have a width that is slightly larger than the width of the valve 30 of the container 16. This allows a valve 30 of a container 16 to be fitted within the valve receiver 28. The valve receiver 28 and aperture 26 can be communicatively connected so that the cooling/heating substance supplied from the valve 30 of the container 16 can be directed to into the indentation 24.

The indentation 24 can also include one or more vents 32. These vents 32 can be used to release excess air or pressurized gas from the exchange chamber 34 by redirecting the excess air or pressurized gas into the bottom portion 14 of the device 10.

The top surface 22 also can include a pivot pin 41 that extends upwards from the top surface 22. The pivot pin 41 can be integrated into the top surface 22, secured to the top surface 22 with an adhesive or screwed into the top surface 22. The pivot pin 41 can be used to secure an item holder 40 to the top surface 22 and allows the item holder 40 to pivot from an open position to a closed position, as shown in FIG. 1 and described more fully below.

The top surface 22 can also be attached to an upper shell 36. The upper shell 36 can be a rounded, hollow shell having a lower circumference 39. The lower circumference 39 of the shell can be attached to a portion of the top surface 22, e.g., one-half of the shell's circumferences can be attached to one-half of the top surface via a connecting wall 35. The connecting wall 35 can be integrated into the upper shell 36 and be connected to the top surface 22 by inserting the wall 35 into a groove 37 etched into the top surface 22. This configuration allows an opening 38 to be formed between the top surface 22 and the upper shell 36. This opening 38 can be used to accept the item holder 40 as it pivots from an open position to a closed position as shown by direction A in FIG. 1. The upper shell 36 can also form an upper portion of the exchange chamber 34. The upper shell 36 also includes a pivot pin receiver 42 on an interior surface. This pivot pin receiver 42 is used to receive a top part of the pivot pin 41.

The pivot pin 41 can be used to secure the item holder 40 in place relative to the top surface 22 and upper shell 36. The item holder 40 can be circular and have a slightly smaller circumference than the circumference of the top surface 22. The item holder 40 also can include a slot 49 for clipping the item holder 40 to the pivot pin 41. The item holder 40 can also include a tab 44 for allowing a user to easily open and close the item holder 22.

The item holder 40 can further include a seat 46 for receiving a composition packet 18. The seat 40 can be a shape that mirrors the shape of the composition packet 18. The composition packet 18 can be a single-use composition packet that can be, e.g., a metallic tray for holding a composition and temporary holds temperature for a short period of time or can be a cardboard wrapped in a metal foil and a small amount of composition can be put on the cardboard before enclosing in the exchange chamber 34. Once a composition packet 18 is seated in the item holder 40, the item holder 40 can be pivoted into the exchange chamber 34 with only an exterior wall 40a of the item holder 40 in view.

The top portion 12 also includes protrusions 52 that extend from an exterior side of the top surface 22 near the circumference of the top surface. These protrusions 52 can align with grooves 54 formed on an interior surface of bottom portion 16 near an opening of the bottom portion 14 ensuring that the top portion 12 and bottom portion 14 are properly aligned.

The sides of the top section 12 can also include spines 56 that extend downwards from the opening. These spines 56 can be used for limiting the amount the top portion can be depressed into the bottom portion 14.

The bottom portion 14 of the device 10 is capable of retaining and supporting the container 16. The bottom portion 14 can be cylindrical having a base 48 and sides 50 extending upwards from the base 48. The sides 50 and base 48 form an open cavity. The interior of the bottom portion 48 can have spines that are of a size for tightly fitting the container 16 when received. In some implementations, the sides 50 can include additional supports for frictionally holding the container with the cavity.

In some implementations, a ring 60 can be snap-fitted with tabs 62 into the bottom portion 14 through apertures 61 located on the sides of the bottom portion 14. This ring 60 can be positioned below the valve 30 of the container 16. In use, when the spines 56 of the top portion 12 make contact with the ring 60, the top portion 12 is preventing from further sliding into the bottom portion 14 thereby preventing the valve 30 from being overly depressed.

In an exemplary example, a container 16 can be inserted into the cavity of the bottom portion 14. The protrusions 52 of the top portion 12 can be aligned with the grooves 54 of the bottom portion 14. The top portion 12, once properly aligned, can be married to the bottom section 14, and as the bottom and top portion 12, 14 are secured to one another with a snap fit, a valve 30 of the container 16 can be inserted into the valve retainer 28. Once in place, the container 16 is ready for use. A user can then pivot the item holder 40 from a closed position to an open position using tab 44. Once opened, the user can insert a packet 18 into the item holder 40 and then close the item holder 40 within the exchange chamber 34. Once closed, the user can depress the top portion 12 which in turn applies pressure to the container valve 30 and releases a pressurized gas or actuates the fan of the electric appliance. The pressurized gas/air enters a lower part of the exchange chamber 34 and, as pressure inside the exchange chamber 34 increases, the gas surrounds the packet 18 thereby cooling/heating the packet 18 to a desired temperature. If too much gas is applied, the gas can be directed downwards into the bottom portion 14 through openings 32 in a bottom portion of the exchange chamber 34. Also excess gas can be directed into the upper shell 36. This allows the gas to be controlled so that excess gas is not forced from any seams in the top portion 12 thereby causing injury to the user. Once a desired temperature is reached, the user can open the item holder 40 and remove the packet 18 for application as needed.

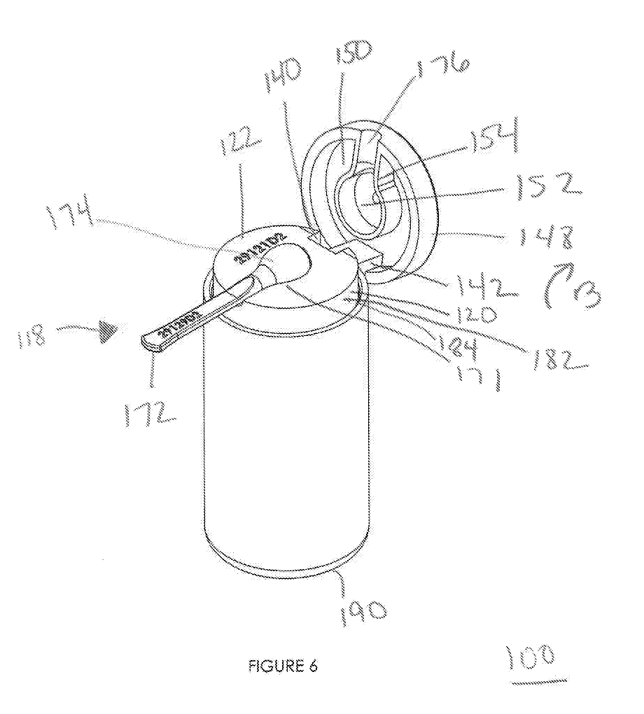

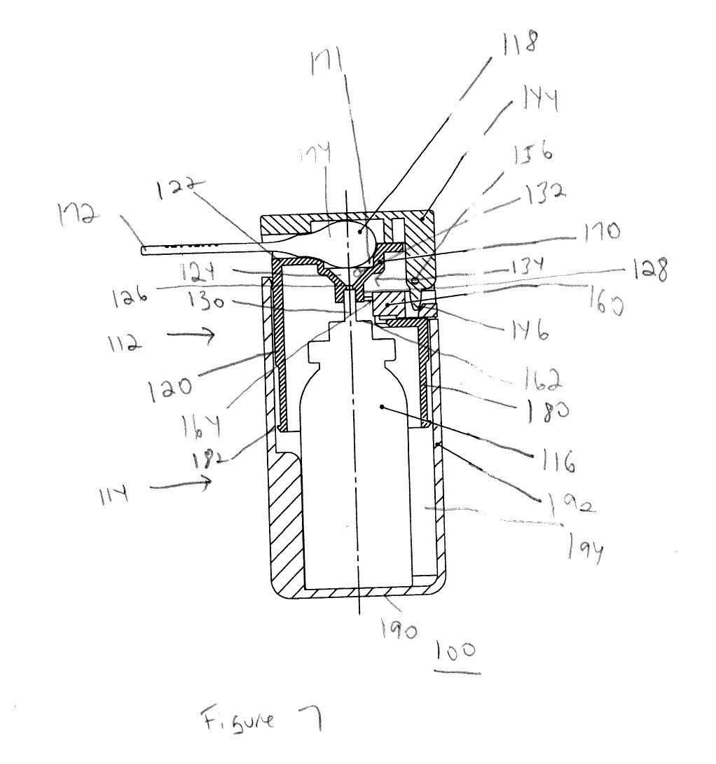

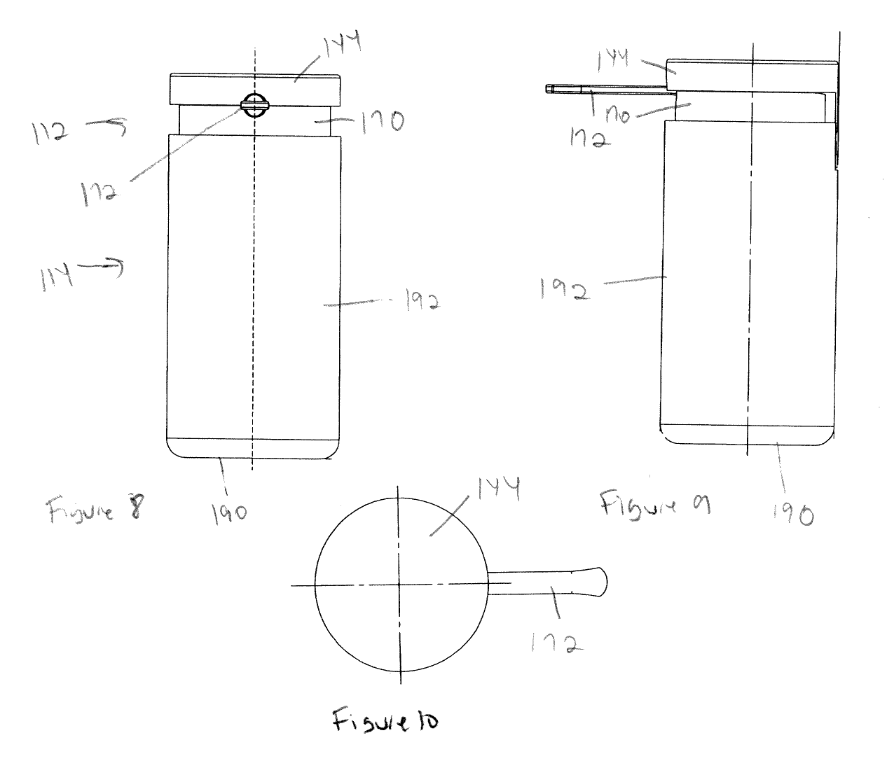

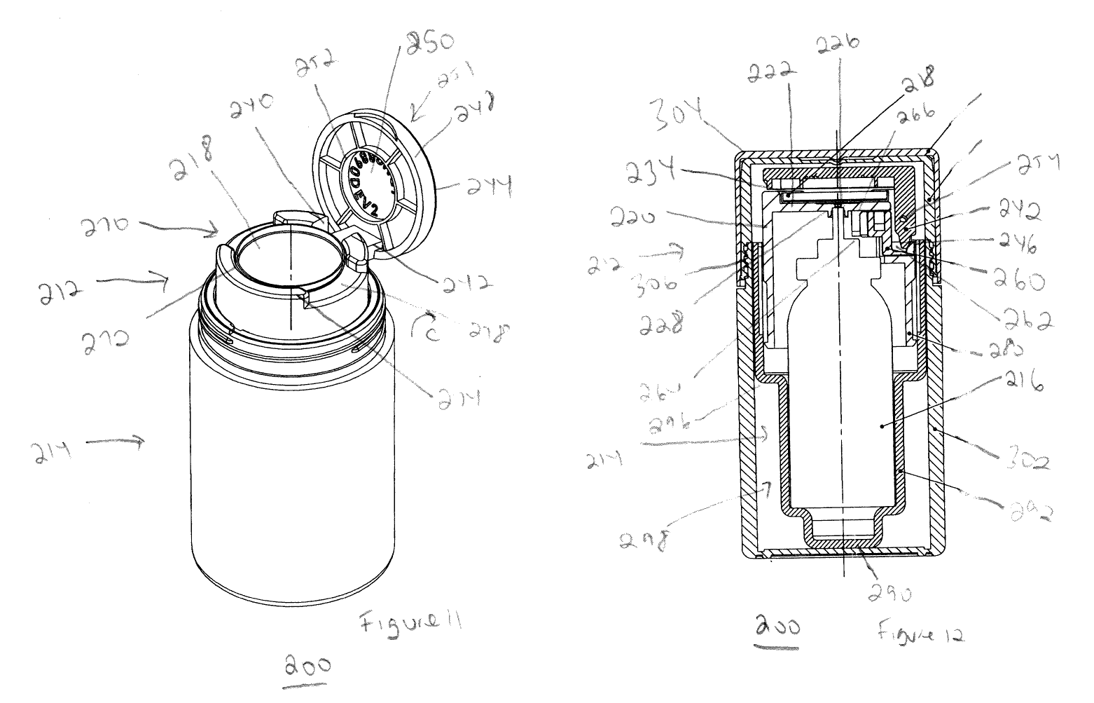

FIGS. 6-10 show another implementation of the disclosed technology. The disclosed technology can be a device 100 that includes a top portion 112 for receiving a pressurized substance from a valve-operated container 116. The container 116 can contain a coolant stored as a liquid gas or the container can be a battery-operated or electric appliance capable of producing cold or hot air. If the container is a cooling container it can be an aerosol container containing a refrigerant. This refrigerant can be, e.g., a hydrocarbon such as 2,3-butane, dimethyl ether or propane or it can be a Freon such as HFC-134a. If the container is a heating container, the container can be a battery-operated electric heating device that allows a heating element and fan to produce a heated gas.

The device 100 can also include a bottom portion 114 for retaining and supporting the container 116. The top portion 112 and bottom portion 114 can be slidably fitted together so to actuate a valve 130 of the container 116 for delivery of the pressurized substance into an exchange chamber 134 located in the top portion 112 thereby cooling or heating an item 118, e.g., an applicator.

The top portion 112 can have a main section 120 that is cylindrical in shape but other shapes and configurations are contemplated. The main section 120 includes a top surface 122. The top surface 122 can be circular, substantially flat and include an indentation 124 at its center. The indentation 124 can be cone-shaped, however, other configurations are contemplated.

The indentation can form a portion of an exchange chamber 134, as will be described more fully below. In the bottom-center of the indentation 124, the indentation can include an aperture 126 that communicatively connects to a valve receiver 128. The valve receiver 128 can protrude downward from a center point of the indentation 124 and be tube shaped. The inner portion of the valve receiver 128 can have a width that is larger than the width of the valve 130 of the container 116. This allows a valve 130 of a container 116 to be fitted within the valve receiver 128. The valve receiver 128 and aperture 126 are communicatively connected so that the cooling/heating substance supplied from the valve 130 of the container 116 can be directed to into the indentation 124.

The indentation 124 can also include one or more vents 132. These vents 132 can be used to release excess air or pressurized gas from the exchange chamber by redirecting the excess air or pressurized gas into the bottom portion 114 of the device 100.

The top surface 122 can further include a cut-out 140 for accepting a lever 142. The lever 142 can be connected to a lid 144 at one end and an extender 146 on the other end. The lid 144 can be round having an exterior wall 148 and a hollow interior 150. The interior 150 can include an upper exchange chamber 152 defined by walls 154. The lid 144 can be moved from a closed position to an open position by the lever 142 in direction B as shown in FIG. 6. The lever 142 is attached to the top portion 112 via a pivot pin 156 integrated into the top portion 112.

The extender 146 of the lever 142 can be communicatively coupled to a valve block 160. That is, the extender 146 can be secured into an opening of the valve block 160 and when the lid 144 is open or closed causes the valve block 160 to move forward and backwards. For example, when the lid 144 is open, the valve block 160 can moved in between a lip 162 of the container 116 and a portion 164 of the top surface 122 thereby inhibiting the valve 130 of the container 116 from being activated. When the lid 144 is closed, the valve block 160 moves away from the lip 162 thereby allowing the valve 130 to freely operate. This configuration is used to prevent the valve 130 from being mistakenly depressed.

The top surface 122 also includes an item holder 170 incorporated therein. The item holder 170 can be a seat 171 for an applicator 118, e.g., the applicator 118 can have a handle 172 and a rounded head 174. The rounded head 174 can be made from a temperature-retaining material, i.e., metal. The seat 171 accommodates the handle 172 and the head 174. The lid 144 also has an area 176 for accepting the handle 172.

The top portion 112 also includes protrusions 182 that extend from an exterior side of the top surface 112 near the circumference of the top surface 122. These protrusions 182 can align with grooves 184 formed on an interior surface of bottom portion 116 near an opening of the bottom portion.

The sides 180 of the top section 122 can extend downwards from the top surface 122 and can include a locking mechanism 181 at a far end. These sides 180 can be used for limiting the amount the top portion 122 can be depressed into the bottom portion 114.

The bottom portion 112 of the device 100 can retain and support the container 116. The bottom portion 112 can be cylindrical having a base 190 and sides 192 extending upwards from the base 190. The sides 192 and base 190 form an open cavity. The interior of the bottom portion 114 can have spines 194 that are of a size for tightly fitting the container 116 when received. In some implementations, the sides 192 can include additional supports (not shown) for frictionally holding the container 116 within the cavity.

In an exemplary example, a container 116 can be inserted into the cavity of the bottom portion 114. Once aligned, the top portion 112 can be inserted into cavity and married with the bottom portion 114. As the bottom and top portion 112, 114 are secured to one another, a valve 130 of the container 116 can be inserted into the valve 130 retainer. Once in place, the container 116 is ready for use. A user then moves the lid 144 from a closed position to an open position. This movement allows the valve block 160 to move in place and restrict the top portion 112 from activating the valve 130 of the container 116. Once opened, the user can insert an applicator 118 into the item holder 140 and then close the lid 144. A user then depresses the top portion 112 which in turn applies pressure to the container valve 130 and releases a pressurized gas. The gas enters the lower gas chamber 134 and, as pressure increase, the gas surrounds the head 14 of the applicator 118 thereby cooling/heating the applicator to a desired temperature. If too much gas is applied, the gas can be directed downwards into the bottom portion 114 through openings 132 in a bottom portion of the chamber 134. Also excess gas can be directed out of the lid 144 through the handle section 176. This allows the gas to be controlled so that excess gas is not forced from any seams in the top portion 112 thereby causing injury to the user. Once a desired temperature is reached, the user can open the item holder 140 and remove the applicator 118 for application for use, as needed.

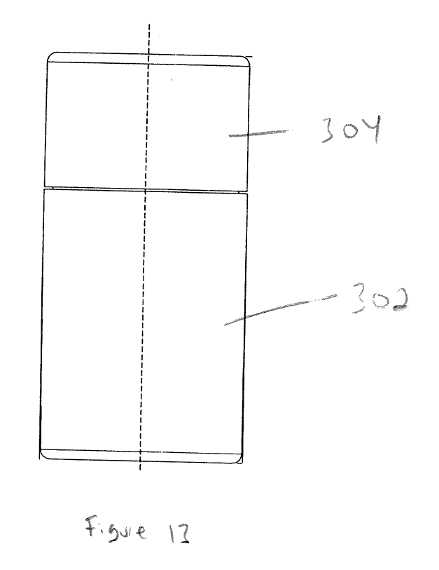

FIGS. 11-13 shows another implementation of the disclosed technology. The disclosed technology can be a device 200 that includes a top portion 212 for receiving a pressurized substance from a valve-operated container 216. The container 216 can contain a coolant stored as a liquid gas or the container can be a battery-operated or electric appliance capable of producing cold or hot air. If the container is a cooling container it can be an aerosol container containing a refrigerant. This refrigerant can be, i.e., a hydrocarbon such as 2,3-butane, dimethyl ether or propane or it can be a Freon, i.e., HFC-134a. If the container is a heating container, the container can be a battery-operated electric heating device that allows a heating element and fan to produce a heated gas.

The device 200 can also include a bottom portion 214 for retaining and supporting the container 216. The top portion 212 and bottom portion 214 can be slidably fitted together so to actuate a valve of the container 216 for delivery of the pressurized substance into an exchange chamber 234 located in the top portion 212 thereby cooling or heating an item, e.g., a cosmetic composition packet or a cosmetic applicator.

The top portion 212 can have a main section 220 that is cylindrical in shape but other shapes and configurations are contemplated. The main section 220 includes a top surface 222. The top surface 222 can be circular and substantially flat.

A center of the top section can include an aperture 226 that communicatively connects to a valve receiver 228. The valve receiver 228 can protrude downward from a center point of the indentation 224 and be tube shaped. The inner portion of the valve receiver 228 can have a width that is larger than the width of the valve 230 of the container 216. This allows a valve 230 of a container 216 to be fitted within the valve receiver 228. The valve receiver 228 and aperture 226 are communicatively connected so that the cooling/heating substance supplied from the valve 230 of the container 216 can be directed into a gas chamber 234.

The top surface 222 can further include a cut-out 240 for accepting a lever 242. The lever 242 can be connected to a lid 244 at one end and have an extender 246 on the other end. The lid 244 can be round having an exterior wall 248 and a hollow interior 250. The interior 250 can include an upper exchange chamber 251 defined by walls 252. The lid 244 can be moved from a closed position to an open position by the lever 242 in direction C as shown in FIG. 11. The lever 242 is attached to the top portion 212 via a pivot pin 254 integrated into the top portion 212.

The extender 246 of the lever 242 can be communicatively coupled to a valve block 260. That is, the extender 246 can be secured into an opening 262 of the valve block 260 and when the lid 244 is open or closed causes the valve block 260 to move forward and backwards. For example, when the lid 244 is open, the valve block 260 can moved in between a lip 264 of the container 216 and the bottom 266 of the top surface 222 thereby inhibiting the valve 230 of the container 216 from being activated. When the lid 244 is closed, the valve block 260 moves away from the lip 264 thereby allowing the valve 230 to freely operate. This configuration is used to prevent the valve 230 from being mistakenly depressed.

The top surface 222 can also include an item holder 270 incorporated therein. The item holder 270 can be a seat 272 for a packet 218. The seat 272 comprises a wall 274 in the shape of the packet 218 and accommodates the packet 218. The wall 274 of the seat 272 can have missing segments 278 that allow gas to escape in a safe manner.

The sides 280 of the top section 212 extend downwards from the top surface 222. These sides 280 can be used for limiting the amount the top portion 212 can be depressed into the bottom portion 214.

The bottom portion 214 of the device retains and supports the container 216. The bottom portion 214 can be a two-tiered cylinder having a base 290 and sides 292 that expand in a single step 296 and extend upwards from the base 290. The sides 292 and base 290 form an open cavity. The bottom cavity 298 is a size to receive the container 216 and the top cavity 300 is of a size to receive the top portion 212. The step 296 can be utilized to stop the sides 280 of top portion 222 from being depressed too far into the bottom portion 214.

The top portion 212 and bottom portion 214 once fitted together can be inserted into an outer casing 302 and closed with a twist-on cap 304 having threads 306.

In an exemplary example, a container 216 can be inserted into the cavity of the bottom portion 214. Once aligned, the top portion 212 can be inserted into cavity and married with the bottom portion 214. As the bottom and top portion 212, 214 are secured to one another, a valve 230 of the container 216 can be inserted into the valve retainer 228. Once in place, the container 216 is ready for use. A user then moves the lid 244 from a closed position to an open position. This movement allows the valve block 260 to move in place and restrict the top portion 212 from activating the valve 230 of the container 216. Once opened, the user can insert a packet 218 into the item holder 270 and then close the lid 244. A user then depresses the top portion 212 which in turn applies pressure to the container valve 230 and releases a pressurized gas. The gas enters the lower gas chamber 234 and, as pressure increase, the gas surrounds the packet 218 thereby cooling/heating the packet 218 to a desired temperature. Excess gas can be directed out of the item holder 270 through the missing segments 278. This allows the gas to be controlled so that excess gas is not forced from any seams in the top portion 212 thereby causing injury to the user. Once a desired temperature is reached, the user can open the lid 244 and remove the packet 218 for application, as needed.

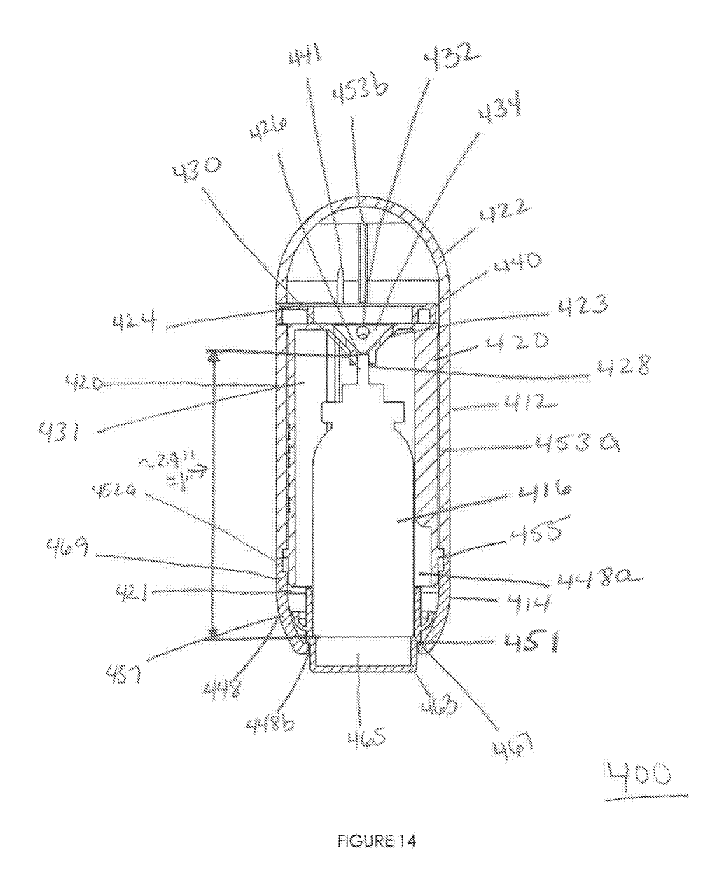

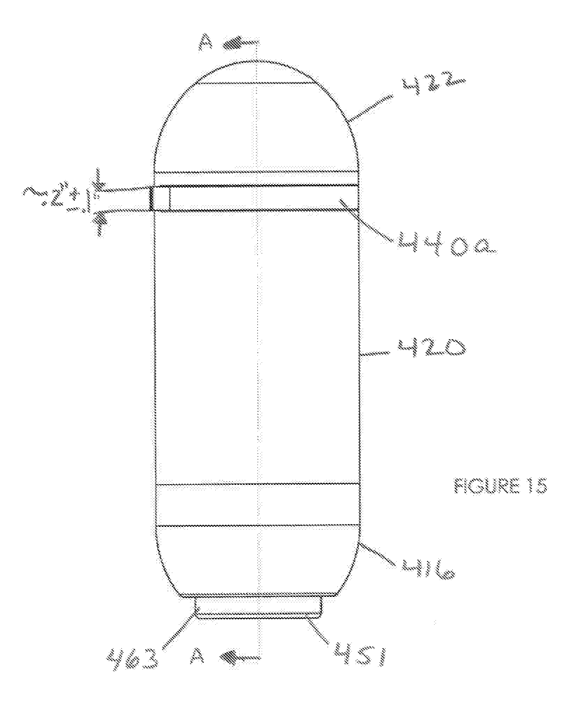

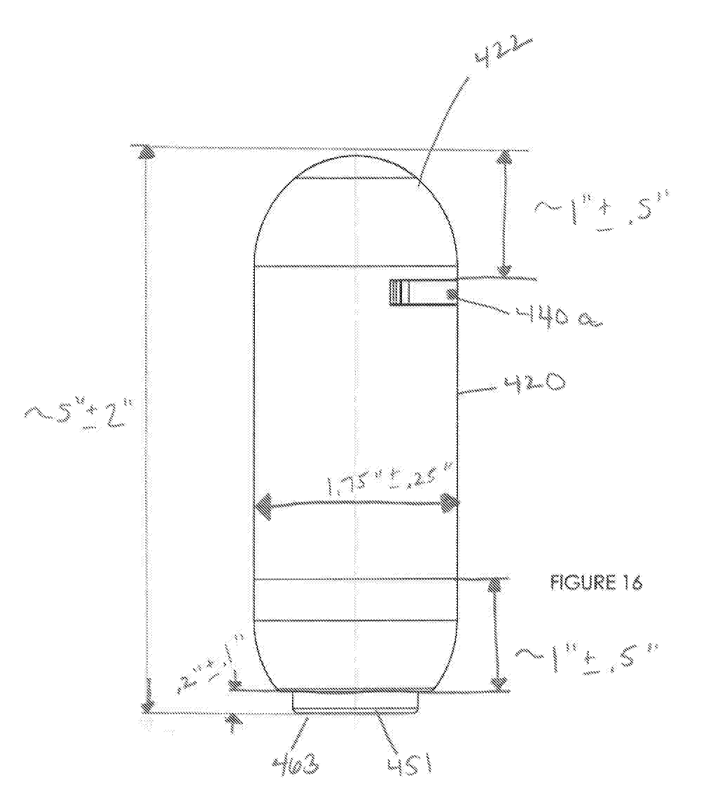

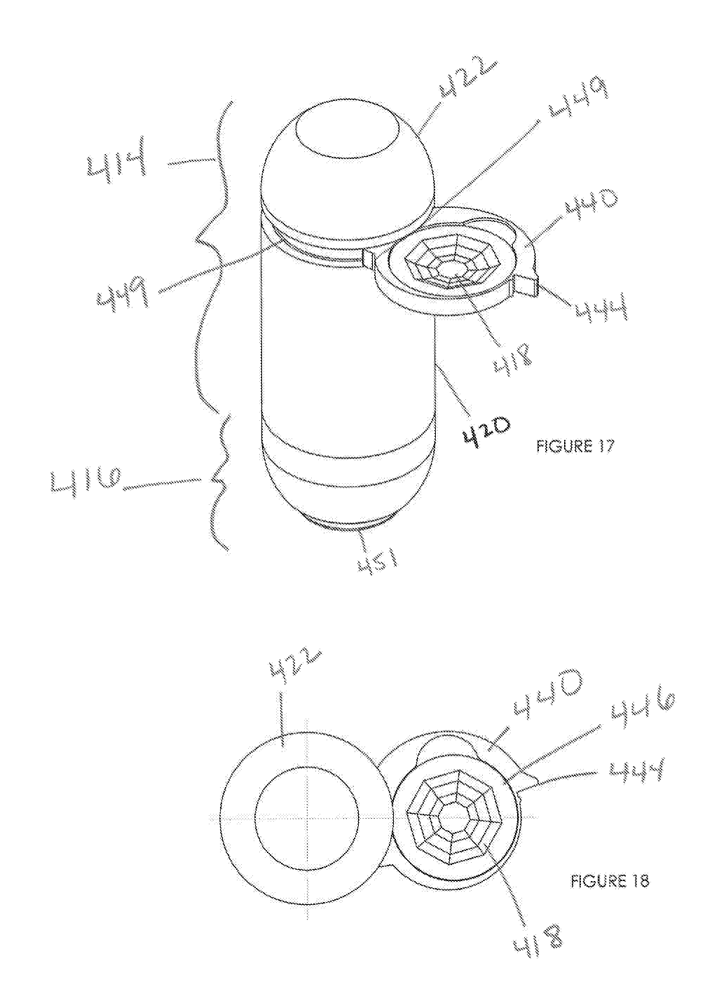

FIGS. 14-18 show an implementation of the disclosed technology. The disclosed technology can be a device 400 for receiving a pressurized substance from a valve-operated container 416. The container 416 can contain a coolant stored as a liquid gas or the container can be a battery-operated or a plugged-in appliance capable of producing pressurized cold or hot air. If the container is a cooling container, it can be an aerosol container containing a refrigerant. This refrigerant can be, e.g., a hydrocarbon, i.e., 2,3-butane, dimethyl ether, propane or a Freon, i.e., HFC-134a. If the container is a heating container, the container can be a battery-operated electric heating device that allows a heating element and fan to produce a pressurized heated gas.

The device 400 can include a top portion 412 and a bottom portion 414 for retaining, supporting and activating the container 416. The top portion 412 and bottom portion 414 can be fitted together so to actuate a valve 430 of the container 416 for delivery of the pressurized substance into an exchange chamber 434 located in the top portion 412 thereby cooling or heating an item 418, e.g., a composition packet.

The top portion 412 can have a main body 420 with a rounded top 422 and an open bottom 421. The main body 420 can be cylindrical in shape and hollow, but other shapes and configurations are contemplated. The rounded top 422 can also be hollow and form an upper portion of the exchange chamber 434, as will be described more fully below.

Within the top portion 412, approximately two-thirds above the bottom opening 421, there can be a surface 423 horizontally positioned within the interior of the top portion 412. The surface 423 can be substantially flat with an indentation 424 at its center. The indentation 424 can be cone-shaped, however, other configurations for the indentation 424 are contemplated. The surface 423 and indentation 424 can form a bottom portion of the exchange chamber 434.

In the bottom-center of the indentation 424, the indentation 424 can include an aperture 426 that communicatively connects to a valve receiver 428. The valve receiver 428 can protrude downward from a center point of the indentation 424 and be, for example, tube shaped. The inner portion of the valve receiver 428 can have a width that is slightly larger than the width of the valve 430 of the container 416. This allows the valve 430 of the container 416 to be fitted within the valve receiver 428. The valve receiver 428 and the aperture 426 can be communicatively connected so that the cooling/heating substance supplied from the valve 430 of the container 416 can be directed to into the indentation 424.

The indentation 424 can also include one or more vents 432. These vents 432 can be used to release excess air or pressurized gas from the exchange chamber 434 by redirecting the excess air or pressurized gas into a space 431 located below the surface 423.

The surface 423 also can include a pivot pin 441 that extends upwards from the surface 423. The pivot pin 441 can be positioned into a pivot pin receiver 442 integrated on an interior surface of the rounded top 422 or integrated into the rounded top 422. The pivot pin 441 can be used to secure an item holder 440 within a recess 449 of the top portion 412 with the item holder being positioned slightly above the surface 423. The pivot pin 441 allows the item holder 440 to pivot from an open position to a closed position within and out of the recess 449, as shown in FIG. 14 and described more fully below.

The item holder 440 can be circular and can include a slot 449 for securing the item holder 440 to the pivot pin 441. The item holder 440 can also include a tab 444 for allowing a user to easily open and close the item holder 422.

The item holder 440 can further include a seat 446 for receiving a composition packet 418. The seat 446 can be a shape that mirrors the shape of the composition packet 418. The composition packet 418 can be a single-use composition packet that can be, e.g., a metallic tray for holding a composition and temporary holds temperature for a short period of time or can be a material, e.g., cardboard, wrapped in a metal foil and a small amount of composition can be put on the material before enclosing in the exchange chamber 434. Once a composition packet 418 is seated in the item holder 440, the item holder 440 can be pivoted into the exchange chamber 434 via the recess 449 having only an exterior wall 440a of the item holder 440 in view.

The top portion 412 can also include two sets of interior spines 453a, 453b. The first set of interior spines 453a can be positioned on the bottom part of the top portion and run vertical. The first set of interior spines 453a can be of a size for fitting and supporting the container 416 in the top portion. The second set of interior spines 453b can be positioned within the rounded top 422 and run vertical within a bend with the rounded top 422. Both sets of the interior spines 453a, 453b add to the structural integrity of the top portion 412.

The top portion 412 can also include a lip 255 and attachment threads 452a positioned at a bottom of the top portion 412. These attachment threads 452a are used for attaching to the bottom portion 414, as will be described more fully below.

The bottom portion 414 of the device 400 is capable of retaining and supporting the container 416. The bottom portion 414 can be a base 448 having an open top 448a and open bottom 448b and an actuator 451 movably positioned in the open bottom 448b of the base 448. The actuator 451 can have a flared section 457. The flared section 457 can have upper and lower portions 457a, 457b, respectively. The lower portion flared section 457b is designed so that a bottom 459 of the actuator 451 can be movably positioned in the opening 448b of the base 448 allowing a majority of the actuator 451 to be positioned within the base 448. The upper portion flared section 457a is designed so that projections 461 of the base 448 can make contact with the upper portion flared section 457a so that the actuator 451 can be limited in the amount the actuator 451 can be depressed into the base 448.

The actuator can further include a seat 467 for receiving the container 416. The seat 267 can be positioned slightly above a bottom wall 463 of the actuator so as to create a space 465 beneath the container. The space 465 can be used to create an insulating distance between the bottom of the container 416 and the bottom of the actuator 451 so that if an activated container freezes/overheats it does not freeze/burn the bottom wall of the actuator 451.

The bottom portion 414 can also include an upper interior wall 469 having attachment threads 452b positioned at a top of the bottom portion 414. The upper interior wall and the lip 255 of the top portion align allowing the attachment threads 452a, 452b to mate so as to secure the top portion 412 to the bottom portion 414.

In an exemplary example, a container 416 can be inserted into the seat 267 of the bottom portion 414. The container 416 can then be aligned with the opening of the top portion 412 and inserted into the top portion. A valve 430 of the container 416 can be inserted into the valve retainer 428. Once properly aligned, the bottom portion 416 and the top portion 414 are secured to one another with the attachment threads 452a, 452b. Once secured, the container 416 is ready for use.

In use, a user can pivot the item holder 440 from a closed position to an open position using tab 444. Once opened, the user can insert a packet 418 into the item holder 440 and then close the item holder 440 within the exchange chamber 434. Once closed, the user can depress the actuator 451 upward which in turn applies pressure to the container valve 430 and releases a pressurized gas or actuates the fan of the electric appliance. The pressurized gas/air enters a lower part of the exchange chamber 434 and, as pressure inside the exchange chamber 434 increases, the gas surrounds the packet 418 thereby cooling/heating the packet 418 to a desired temperature. If too much gas is applied, the gas can be directed downwards into the bottom portion 414 through openings 432 in a bottom portion of the exchange chamber 434. Also excess gas can be directed into the rounded top. This pressure relief allows the gas to be controlled so that excess gas is not forced from any seams in the device 400 thereby causing injury to the user. Once a desired temperature is reached, the user can remove pressure from the actuator and open the item holder 440. The packet 418 can then be removed and applied as needed.

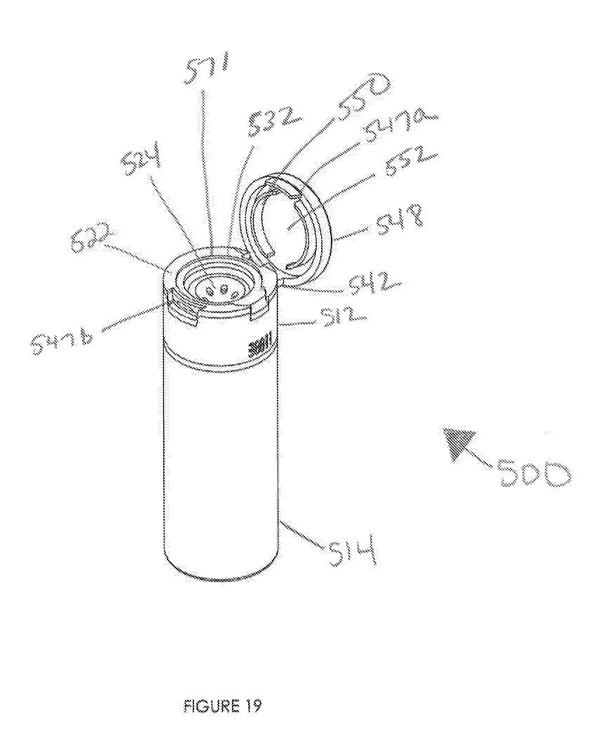

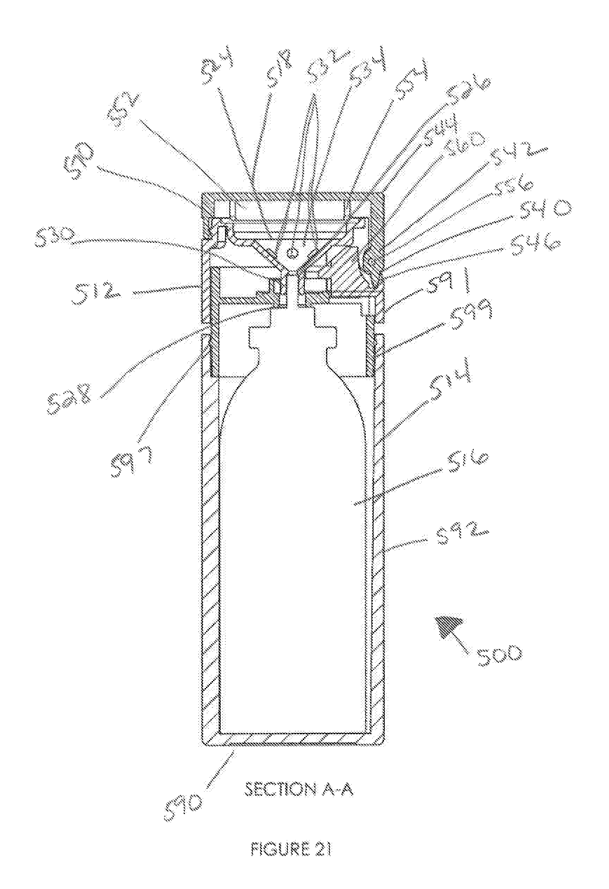

FIGS. 19-21 show another implementation of the disclosed technology. The disclosed technology can be a device 500 that includes a top portion 512 for receiving a pressurized substance from a valve-operated container 516. The container 516 can contain a coolant stored as a liquid gas or the container can be a battery-operated or electric appliance capable of producing cold or hot air. If the container is a cooling container it can be an aerosol container containing a refrigerant. This refrigerant can be, e.g., a hydrocarbon such as 2,3-butane, dimethyl ether or propane or it can be a Freon such as HFC-134a. If the container is a heating container, the container can be a battery-operated electric heating device that allows a heating element and fan to produce a heated gas.

The device 500 can also include a bottom portion 514 for retaining and supporting the container 516. The top portion 512 and bottom portion 514 can be fitted together so to actuate a valve 530 of the container 516 for delivery of the pressurized substance into an exchange chamber 534 located in the top portion 512 thereby cooling or heating an item 518, e.g., an applicator or a cosmetic packet. The top portion 512 and bottom portion 514 can have a combined height of H₁, where H₁ can vary between 1 and 8 inches.

The top portion 512 can have a main section 520 that is cylindrical in shape but other shapes and configurations are contemplated. The main section 520 includes a top surface 522. The top surface 522 can be circular and include an indentation 524 at its center. The indentation 524 can be cone-shaped, however, other configurations are contemplated.

The indentation 524 can form a portion of an exchange chamber 534, as will be described more fully below. In the bottom-center of the indentation 524, the indentation can include an aperture 526 that communicatively connects to a valve receiver 528. The valve receiver 528 can protrude downward from a center point of the indentation 524 and be tube shaped. The inner portion of the valve receiver 528 can have a width that is larger than the width of the valve 530 of the container 516. This allows a valve 530 of a container 516 to be fitted within the valve receiver 528. The valve receiver 528 and aperture 526 are communicatively connected so that the cooling/heating substance supplied from the valve 530 of the container 516 can be directed to into the indentation 524 of the exchange chamber 534.

The indentation 524 can also include one or more vents 532. These vents 532 can be used to release excess air or pressurized gas from the exchange chamber 534 by redirecting the excess air or pressurized gas into the bottom portion 514 of the device 500.

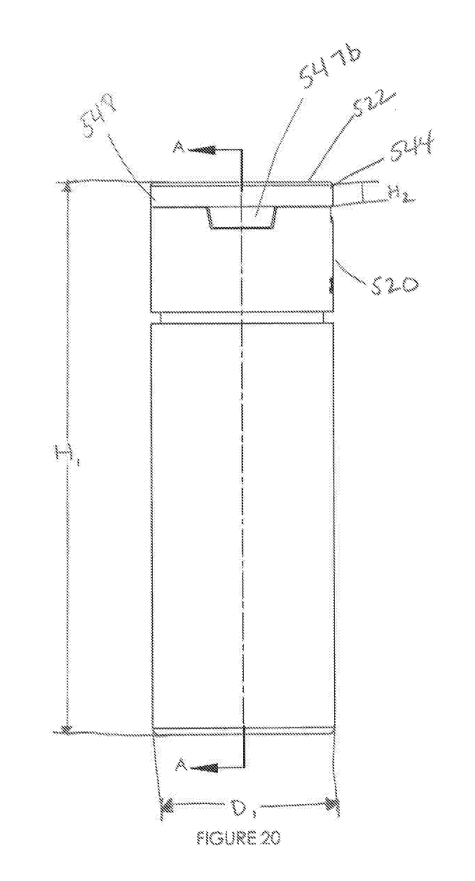

The top surface 522 can further include a cut-out 540 for accepting a lever 542 of a lid 544 at one end and an extender 546 on the other end. The lid 544 can be round having an exterior wall 548, a hollow interior 550, locking mechanism 547a, 547b and a height—H₂ where H₂ can be 1/16 to ¼ inches. The interior 550 can include an upper exchange chamber 552 defined by walls 554. The lid 544 can be moved from a closed position (shown in FIG. 20) to an open position (shown in FIG. 19) by the lever 542. The lever 542 can be attached to the top portion 512 via a pivot pin 556 integrated into the top portion 512.

The extender 546 of the lever 542 can be communicatively coupled to a valve block 560 via extender 654. That is, the extender 546 can be pivot with the cut-out to move the valve block 560 so that when the lid 544 is open or closed the valve block 560 can move forward and backwards. For example, when the lid 544 is open, the valve block 560 can moved in between a lip 562 of the container 516 and a portion 564 of the top surface 522 thereby inhibiting the valve 530 of the container 516 from being activated. When the lid 544 is closed, the valve block 560 moves away from the lip 562 thereby allowing the valve 530 to freely operate. This configuration is used to prevent the valve 530 from being mistakenly depressed when the lid is in an opened position.

The top portion 512 can also include a rotational stopping device 591 that enables the top portion 512 to rotate and cause a second blocker to interfere with the depressing function of the device 500 so that when the lid is closed and the rotational stopping device 591 is in a locked position, the device 500 cannot be actuated. This configuration is used to prevent the valve 530 from being mistakenly depressed when the lid is in a closed position but use was accidental, e.g. device is in a travel bag and any use was inadvertent.

The top surface 522 also includes an item holder 570 incorporated therein. The item holder 570 can be a seat 571 for a cosmetic packet 518, e.g., the composition packet 518 can be a single-use composition packet that can be, e.g., a metallic tray for holding a composition and temporary holds temperature for a short period of time or can be a cardboard wrapped in a metal foil and a small amount of composition can be put on the cardboard before enclosing in the exchange chamber 534.

The bottom portion 512 of the device 500 can retain and support the container 116. The bottom portion 512 can be cylindrical having a base 590 and sides 592 extending upwards from the base 590. The sides 592 and base 590 form an open cavity.

In an exemplary example, a container 516 can be inserted into the cavity of the bottom portion 514. Once aligned, the top portion 512 can be inserted into cavity and be removably locked to the bottom portion 514 with locking mechanisms 597, 599. As the bottom and top portion 512, 514 are secured to one another, a valve 530 of the container 516 can be inserted into the valve 530 retainer. Once in place, the container 516 is ready for use.

To use, a user twists the rotational stopping device 591 and unlocks the device 500. The user then moves the lid 544 from a closed position to an open position. This movement allows the valve block 560 to move in place and restrict the top portion 512 from activating the valve 530 of the container 516. Once opened, the user can insert an item 518 into the item holder 540 and then close the lid 544. A user then depresses the top portion 512 which in turn applies pressure to the container valve 530 and releases a pressurized gas. The gas enters the lower gas chamber 534 and, as pressure increases, the gas surrounds the item 518 thereby cooling/heating the item 518 to a desired temperature. If too much gas is applied, the gas can be directed downwards into the bottom portion 514 through openings 532 in a bottom portion of the chamber 534. This allows the gas to be controlled so that excess gas is not forced from any seams in the top portion 512 thereby causing injury to the user. Once a desired temperature is reached, the user can open the item holder 540 and remove the cooled item 518 for application, as needed.

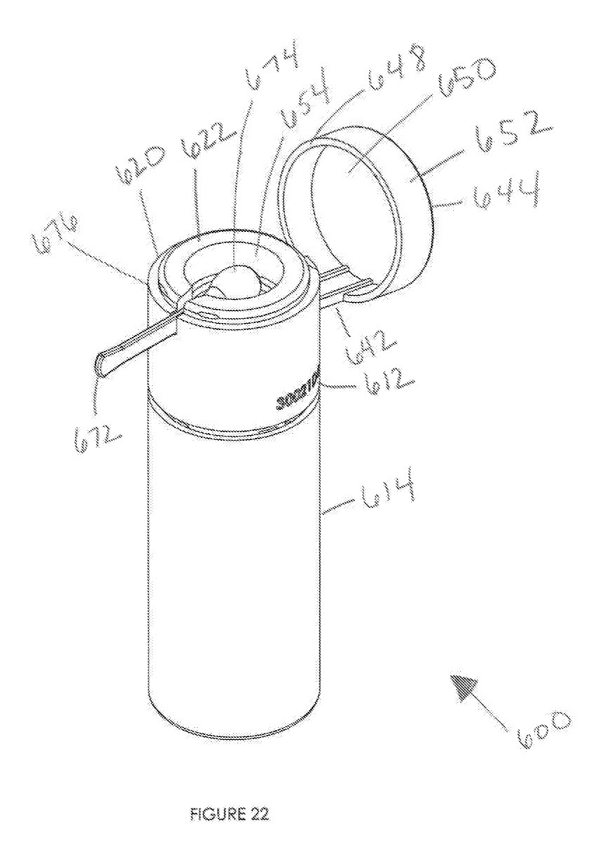

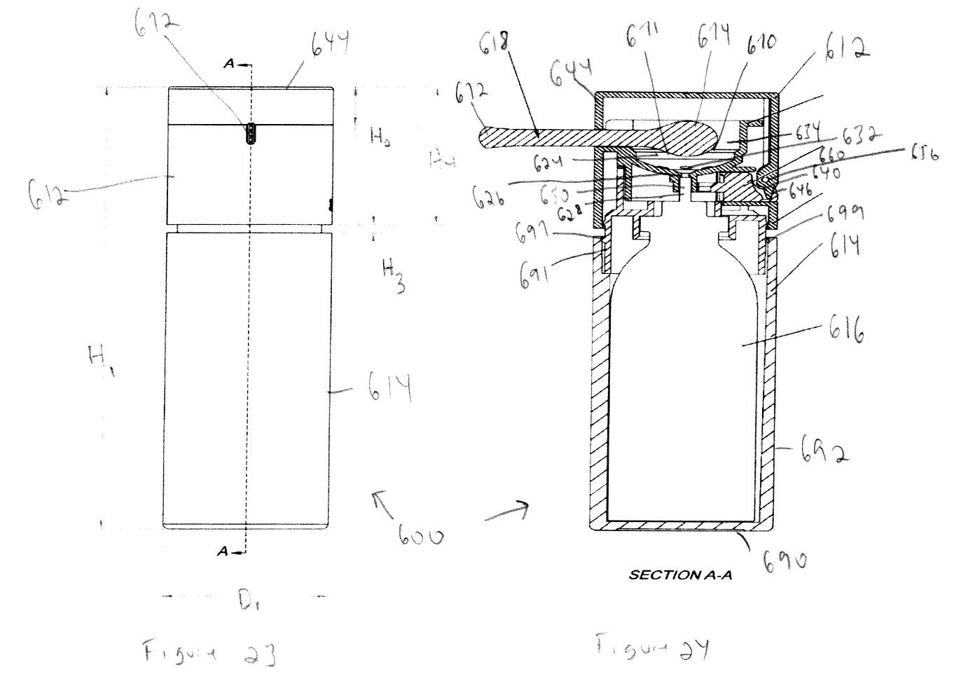

FIGS. 22-24 show another implementation of the disclosed technology. The disclosed technology can be a device 600 that includes a top portion 612 for receiving a pressurized substance from a valve-operated container 616. The container 616 can contain a coolant stored as a liquid gas or the container can be a battery-operated or electric appliance capable of producing cold or hot air. If the container is a cooling container it can be an aerosol container containing a refrigerant. This refrigerant can be, e.g., a hydrocarbon such as 2,3-butane, dimethyl ether or propane or it can be a Freon such as HFC-134a. If the container is a heating container, the container can be a battery-operated electric heating device that allows a heating element and fan to produce a heated gas.

The device 600 can also include a bottom portion 614 for retaining and supporting the container 616. The top portion 612 and bottom portion 614 can be removably fitted together with locking mechanisms 697, 699 so to actuate a valve 630 of the container 616 for delivery of the pressurized substance into an exchange chamber 634 located in the top portion 612 thereby cooling or heating an item 618, e.g., an applicator or a cosmetic packet. The top portion 612 and bottom portion 614 can have a combined height of H₁ and a diameter of D₁.

The top portion 612 can have a main section 620 that is cylindrical in shape but other shapes and configurations are contemplated. The main section 620 includes a top surface 622. The top surface 622 can be circular and include an indentation 624 at its center. The indentation 624 can be cone-shaped, however, other configurations are contemplated.

The indentation can form a portion of an exchange chamber 634, as will be described more fully below. In the bottom-center of the indentation 624, the indentation can include an aperture 626 that communicatively connects to a valve receiver 628. The valve receiver 628 can protrude downward from a center point of the indentation 624 and be tube shaped. The inner portion of the valve receiver 628 can have a width that is larger than the width of the valve 630 of the container 616. This allows a valve 630 of a container 616 to be fitted within the valve receiver 628. The valve receiver 628 and aperture 626 are communicatively connected so that the cooling/heating substance supplied from the valve 630 of the container 616 can be directed to into the indentation 624.

The indentation 624 can also include one or more vents 632. These vents 632 can be used to release excess air or pressurized gas from the exchange chamber 634 by redirecting the excess air or pressurized gas into the bottom portion 614 of the device 600.

The top surface 622 can further include a cut-out 640 for accepting a lever 642 of a lid 644 at one end and an extender 646 on the other end. The lid 644 can be round having an exterior wall 648, a hollow interior 650, and a height—H₂. The interior 650 can include an upper exchange chamber 652 defined by walls 654. The lid 644 can be moved from a closed position (shown in FIG. 23) to an open position (shown in FIG. 22) by the lever 642. The lever 642 can be attached to the top portion 512 via a pivot pin 656 integrated into the top portion 612.

The extender 646 of the lever 642 can be communicatively coupled to a valve block 660 via extender 646 so that when the lid 644 is open or closed the valve block 660 can move forward and backwards. For example, when the lid 644 is open, the valve block 660 can moved in between a lip 662 of the container 616 and a portion 664 of the top surface 622 thereby inhibiting the valve 630 of the container 616 from being activated. When the lid 644 is closed, the valve block 660 moves away from the lip 662 thereby allowing the valve 630 to freely operate. This configuration is used to prevent the valve 630 from being mistakenly depressed when the lid is in an opened position.

The top portion 612 can also include a rotational stopping device 691 that enables the top portion 612 to rotate and cause a second blocker to interfere with the depressing function of the device 600 so that when the lid is closed and the rotational stopping device 691 is in a locked position, the device 600 cannot be actuated. This configuration is used to prevent the valve 630 from being mistakenly depressed when the lid is in a closed position, e.g. device is in a travel bag and any use was inadvertent. A distance H₃ between the top portion 12 and upper edge of the bottom section 614 is used to inhibit the depth the top portion 612 can actuate the valve 630.

The top surface 622 also includes an item holder 670 incorporated therein. The item holder 670 can be a seat 671 for an applicator 618, e.g., the applicator 618 can have a handle 672 and a rounded head 674. The rounded head 674 can be made from a temperature-retaining material, i.e., metal. The seat 671 accommodates the handle 672 and the head 674. The lid 644 also has an area 676 for accepting the handle 672.

The bottom portion 612 of the device 600 can retain and support the container 616. The bottom portion 612 can be cylindrical having a base 690 and sides 692 extending upwards from the base 690. The sides 692 and base 690 form an open cavity.

In an exemplary example, a container 616 can be inserted into the cavity of the bottom portion 614. Once aligned, the top portion 612 can be inserted into cavity and be removably locked to the bottom portion 614 with locking mechanisms 697, 699. As the bottom and top portion 612, 614 are secured to one another, a valve 630 of the container 616 can be inserted into the valve 630 retainer. Once in place, the container 616 is ready for use.

To use, a user twists the rotational stopping device 691 and unlocks the device 600. The user then moves the lid 644 from a closed position to an open position. This movement allows the valve block 660 to move in place and restrict the top portion 612 from activating the valve 630 of the container 616. Once opened, the user can insert an item 618 into the item holder 640 and then close the lid 644. A user then depresses the top portion 612 which in turn applies pressure to the container valve 630 and releases a pressurized gas. The gas enters the lower gas chamber 634 and, as pressure increase, the gas surrounds the item 618 thereby cooling/heating the item 618 to a desired temperature. If too much gas is applied, the gas can be directed downwards into the bottom portion 614 through openings 632 in a bottom portion of the chamber 634. This allows the gas to be controlled so that excess gas is not forced from any seams in the top portion 612 thereby causing injury to the user. Once a desired temperature is reached, the user can open the item holder 640 and remove the cooled item 618 for application, as needed.

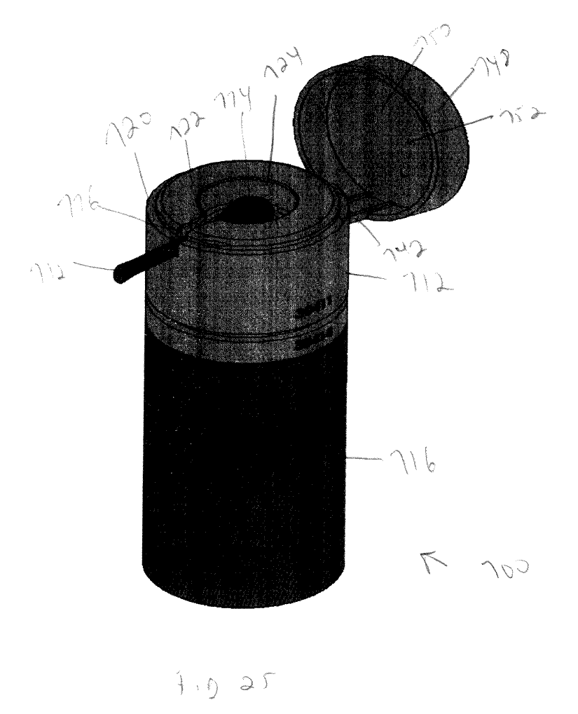

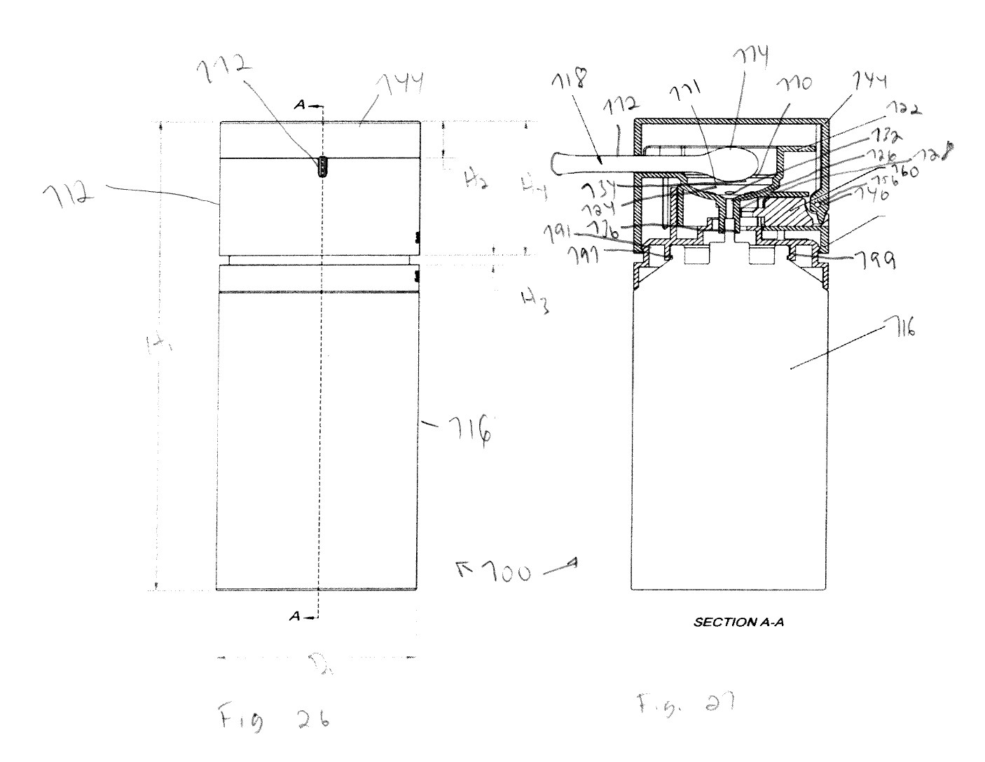

FIGS. 25-28 show another implementation of the disclosed technology. The disclosed technology can be a device 700 that includes a top portion 712 for receiving a pressurized substance from a valve-operated container 716. The container 716 can contain a coolant stored as a liquid gas or the container can be a battery-operated or electric appliance capable of producing cold or hot air. If the container is a cooling container it can be an aerosol container containing a refrigerant. This refrigerant can be, e.g., a hydrocarbon such as 2,3-butane, dimethyl ether or propane or it can be a Freon such as HFC-134a. If the container is a heating container, the container can be a battery-operated electric heating device that allows a heating element and fan to produce a heated gas.

The top portion 712 and the container 716 can be removably fitted together with locking mechanisms 797, 799 so to actuate a valve 730 of the container 716 for delivery of the pressurized substance into an exchange chamber 734 located in the top portion 712 thereby cooling or heating an item 718, e.g., an applicator or a cosmetic packet.

The top portion 712 can have a main section 720 that is cylindrical in shape but other shapes and configurations are contemplated. The main section 720 includes a top surface 722. The top surface 722 can be circular and include an indentation 724 at its center. The indentation 724 can be cone-shaped, however, other configurations are contemplated.

The indentation can form a portion of an exchange chamber 734, as will be described more fully below. In the bottom-center of the indentation 724, the indentation can include an aperture 726 that communicatively connects to a valve receiver 728. The valve receiver 728 can protrude downward from a center point of the indentation 724 and be tube shaped. The inner portion of the valve receiver 728 can have a width that is larger than the width of the valve 730 of the container 716. This allows a valve 730 of a container 716 to be fitted within the valve receiver 728. The valve receiver 728 and aperture 726 are communicatively connected so that the cooling/heating substance supplied from the valve 730 of the container 716 can be directed to into the indentation 724.

The indentation 724 can also include one or more vents 732. These vents 732 can be used to release excess air or pressurized gas from the exchange chamber 734 by redirecting the excess air or pressurized gas into the bottom portion 714 of the device 700.

The top surface 722 can further include a cut-out 740 for accepting a lever 742. The lever 742 can be connected to a lid 744 at one end and an extender 746 on the other end. The lid 744 can be round having an exterior wall 748 and a hollow interior 750. The interior 750 can include an upper exchange chamber 752. The lid 744 can be moved from a closed position to an open position by the lever 742. The lever 742 can be attached to the top portion 712 via a pivot pin 756 integrated into the top portion 712.

The extender 746 of the lever 742 can be communicatively coupled to a valve block 760. That is, the extender 746 can be secured into an opening of the valve block 760 and when the lid 744 is open or closed causes the valve block 760 to move forward and backwards. For example, when the lid 744 is open, the valve block 760 can moved in between a lip 762 of the container 716 and a portion 764 of the top surface 722 thereby inhibiting the valve 730 of the container 716 from being activated. When the lid 744 is closed, the valve block 760 moves away from the lip 762 thereby allowing the valve 730 to freely operate. This configuration is used to prevent the valve 730 from being mistakenly depressed when opened.

The top portion 712 can also include a rotational stopping device 791 that enables the top portion 712 to rotate and cause a blocker to interfere with the depressing function of the device 700 so that when the lid is closed and the rotational stopping device 691 is in a locked position, the device 700 cannot be actuated.

The top surface 722 also includes an item holder 770 incorporated therein. The item holder 770 can be a seat 771 for an applicator 718, e.g., the applicator 718 can have a handle 772 and a rounded head 774. The rounded head 774 can be made from a temperature-retaining material, i.e., metal. The seat 771 accommodates the handle 772 and the head 774. The lid 744 also has an area 776 for accepting the handle 772.

In an exemplary example, a container 716 can be removably secured to a top portion 712. As the container 716 and top portion 712 are secured to one another, a valve 730 of the container 716 can be inserted into the valve 730. Once in place, the container 716 is ready for use.

To use, a user twists the rotational stopping device 791 and unlocks the device 700. The user then moves the lid 744 from a closed position to an open position. This movement allows the valve block 760 to move in place and restrict the top portion 712 from activating the valve 730 of the container 716. Once opened, the user can insert an item 718 into the item holder 740 and then close the lid 744. A user then depresses the top portion 712 which in turn applies pressure to the container valve 730 and releases a pressurized gas. The gas enters the lower gas chamber 734 and, as pressure increase, the gas surrounds the item 718 thereby cooling/heating the item 718 to a desired temperature. If too much gas is applied, the gas can be directed downwards into the bottom portion 714 through openings 732 in a bottom portion of the chamber 734. This allows the gas to be controlled so that excess gas is not forced from any seams in the top portion 712 thereby causing injury to the user. Once a desired temperature is reached, the user can open the item holder 740 and remove the cooled item 718 for application, as needed.

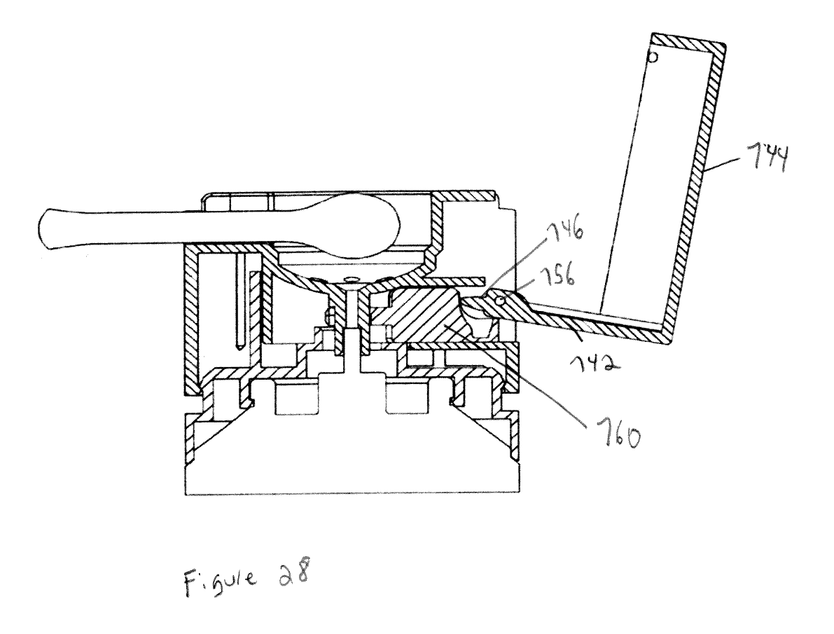

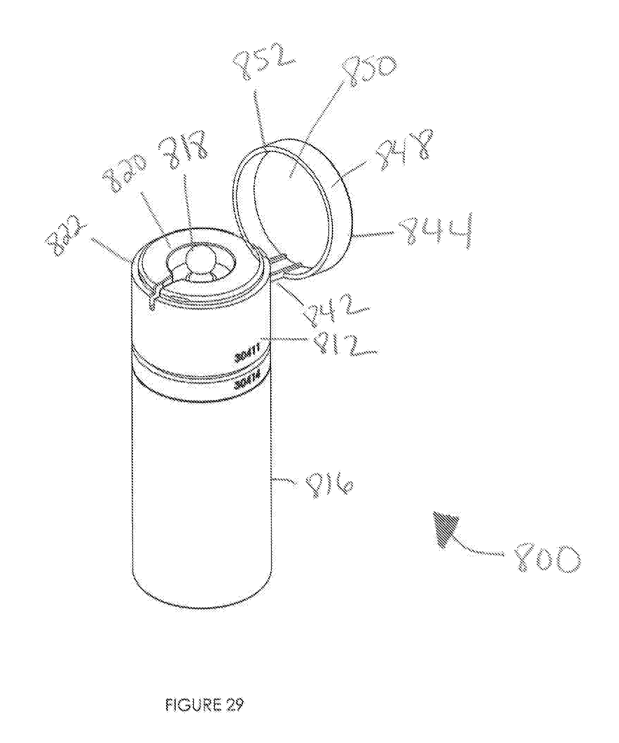

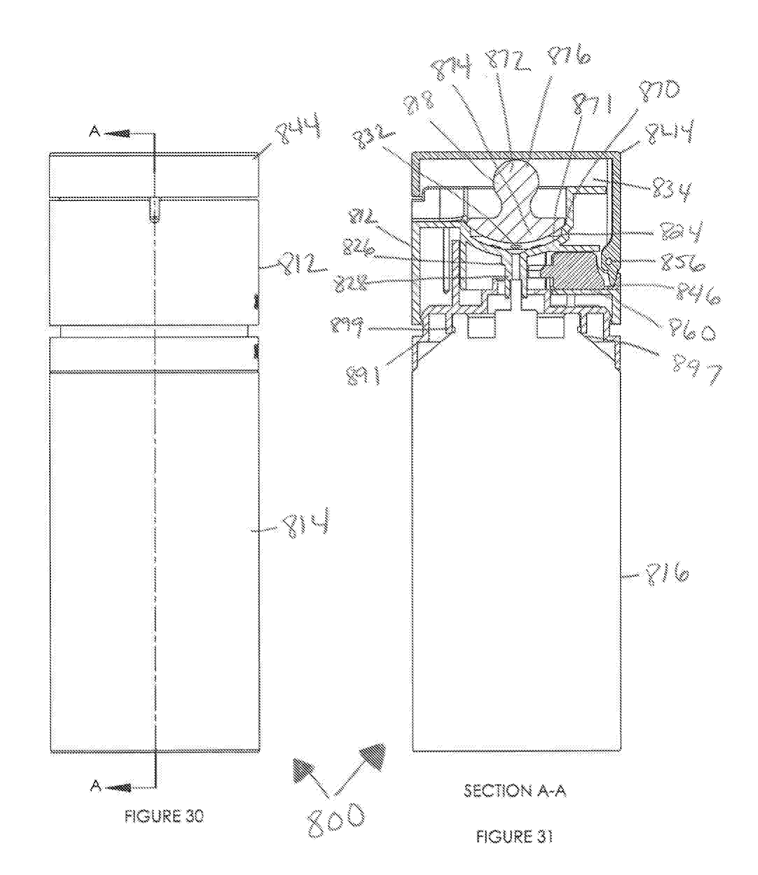

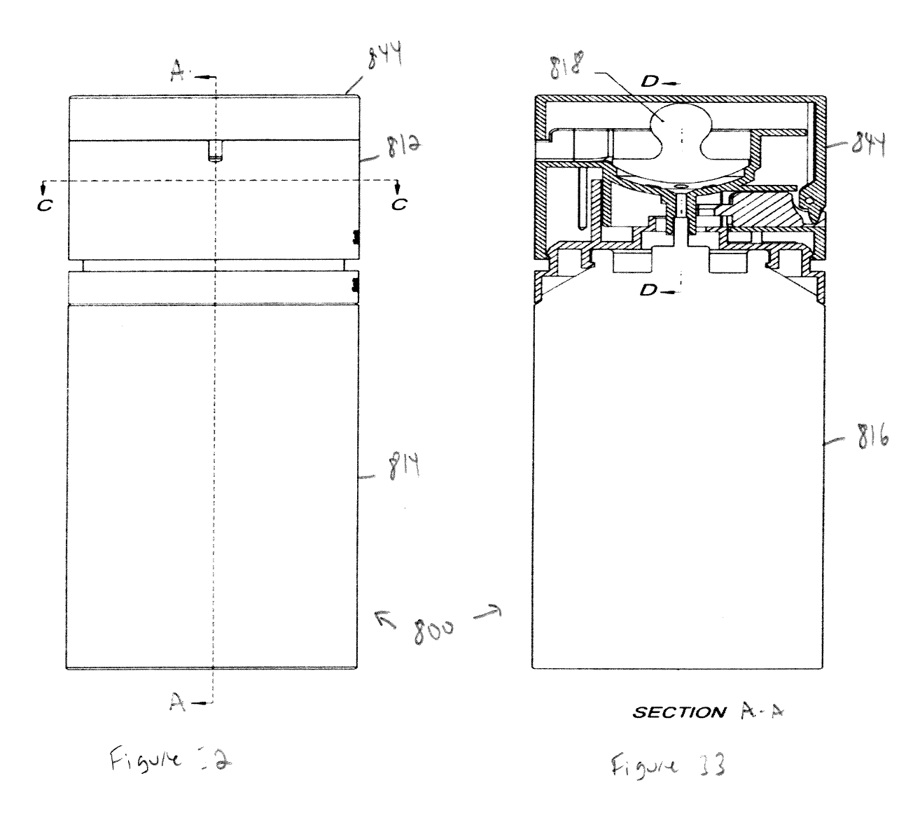

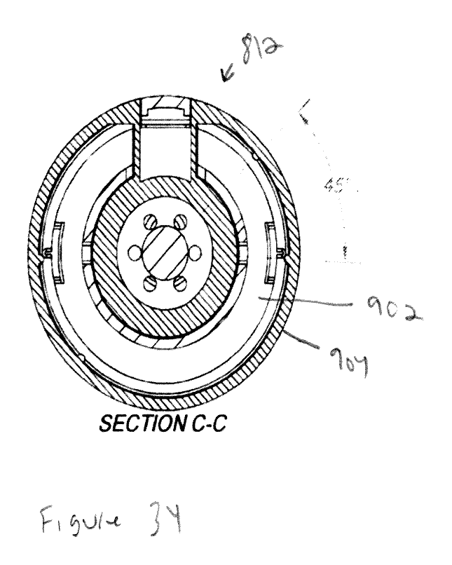

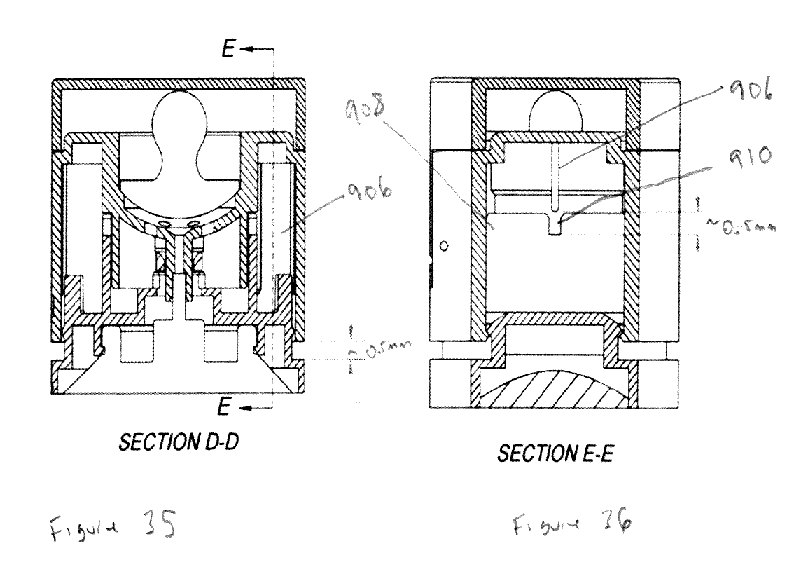

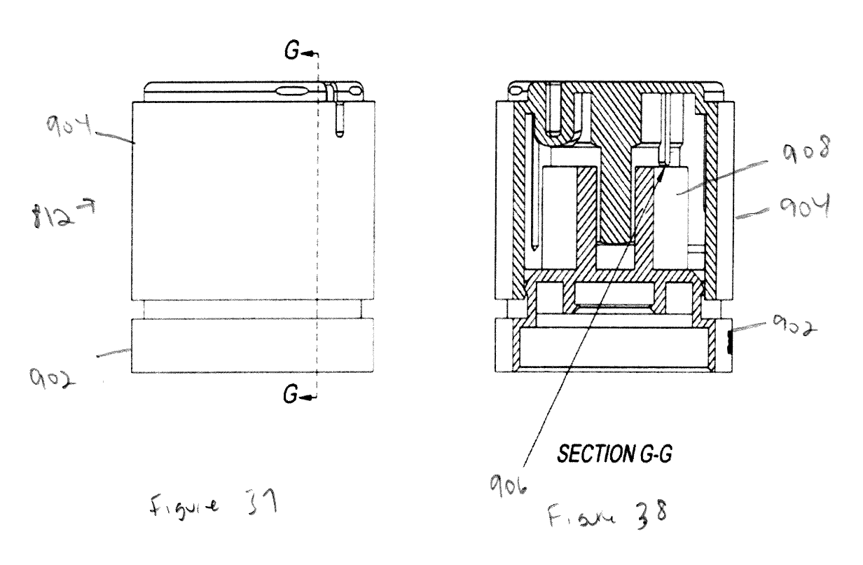

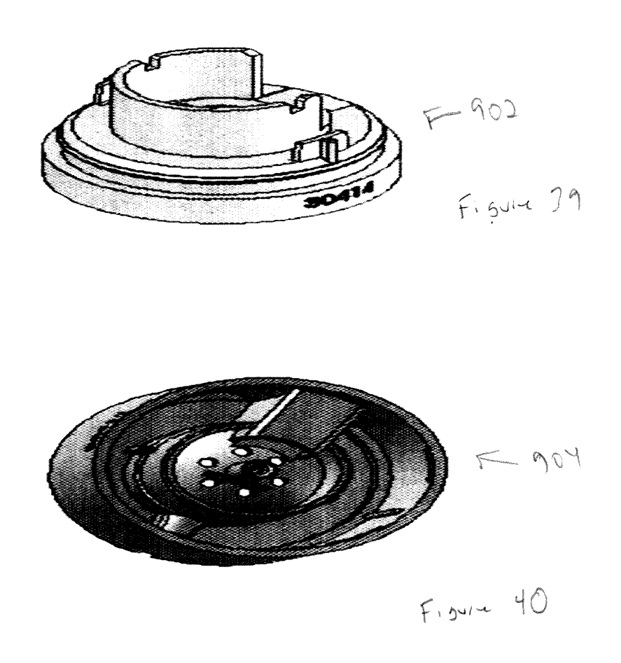

FIGS. 29-40 show another implementation of the disclosed technology. The disclosed technology can be a device 800 that includes a top portion 812 for receiving a pressurized substance from a valve-operated container 816. The container 816 can contain a coolant stored as a liquid gas or the container can be a battery-operated or electric appliance capable of producing cold or hot air. If the container is a cooling container it can be an aerosol container containing a refrigerant. This refrigerant can be, e.g., a hydrocarbon such as 2,3-butane, dimethyl ether or propane or it can be a Freon such as HFC-134a. If the container is a heating container, the container can be a battery-operated electric heating device that allows a heating element and fan to produce a heated gas.

The top portion 812 and the container 816 can be removably fitted together with locking mechanisms 897, 899 so to actuate a valve 830 of the container 816 for delivery of the pressurized substance into an exchange chamber 834 located in the top portion 812 thereby cooling or heating an item 818, e.g., an applicator or a cosmetic packet.

The top portion 812 can have a main section 820 that is cylindrical in shape but other shapes and configurations are contemplated. The main section 820 includes a top surface 822. The top surface 822 can be circular, substantially flat and include an indentation 824 at its center. The indentation 824 can be cone-shaped, however, other configurations are contemplated.

The indentation can form a portion of an exchange chamber 834, as will be described more fully below. In the bottom-center of the indentation 824, the indentation can include an aperture 826 that communicatively connects to a valve receiver 828. The valve receiver 828 can protrude downward from a center point of the indentation 824 and be tube shaped. The inner portion of the valve receiver 828 can have a width that is larger than the width of the valve 830 of the container 816. This allows a valve 830 of a container 816 to be fitted within the valve receiver 828. The valve receiver 828 and aperture 826 are communicatively connected so that the cooling/heating substance supplied from the valve 830 of the container 816 can be directed to into the indentation 824.

The indentation 824 can also include one or more vents 832. These vents 832 can be used to release excess air or pressurized gas from the exchange chamber 834 by redirecting the excess air or pressurized gas into the bottom portion 814 of the device 800.

The top surface 822 can further include a cut-out 840 for accepting a lever 842. The lever 842 can be connected to a lid 844 at one end and an extender 846 on the other end. The lid 844 can be round having an exterior wall 848 and a hollow interior 850. The interior 850 can include an upper exchange chamber 852. The lid 844 can be moved from a closed position to an open position by the lever 842. The lever 842 can be attached to the top portion 812 via a pivot pin 856 integrated into the top portion 812.

The extender 846 of the lever 842 can be communicatively coupled to a valve block 860. That is, the extender 846 can be secured into an opening of the valve block 860 and when the lid 844 is open or closed causes the valve block 860 to move forward and backwards. For example, when the lid 844 is open, the valve block 860 can moved in between a lip 862 of the container 816 and a portion 864 of the top surface 822 thereby inhibiting the valve 830 of the container 816 from being activated. When the lid 844 is closed, the valve block 860 moves away from the lip 862 thereby allowing the valve 830 to freely operate. This configuration is used to prevent the valve 830 from being mistakenly depressed when opened.

The top portion 812 can also include a rotational stopping device 891 that enables the top portion 812 to rotate and cause a blocker to interfere with the depressing function of the device 800 so that when the lid is closed and the rotational stopping device 891 is in a locked position, the device 800 cannot be actuated. As shown in FIGS. 34-38, the top portion 812 can have a lower section 902 and an upper section 904. The upper section 904 and the lower section 902 are movably attached to one another and can rotate relative to each other. In some implementations, as shown in FIG. 34, the rotation can be 45 degrees but other angles of rotation are contemplated. In use, the top section includes a rib 906 that slides relative to wall 908. As the top section is rotated, the rib lines up with opening 910. Once the rib 906 and groove 910 are aligned, the device can be depressed for use.

The top surface 822 also includes an item holder 870 incorporated therein. The item holder 870 can be a seat 871 for an applicator 818, e.g., the applicator 818 can have a handle 872 and a rounded bottom 874. The rounded bottom 874 can be made from a temperature-retaining material, i.e., metal. The seat 871 accommodates the handle 872 and the bottom 874. The lid 844 also has an area 876 for accepting the handle 872.

In an exemplary example, a container 816 is removably locked to the top portion 812 with locking mechanisms 897, 899. As the container 816 and top portion 812 are secured to one another, a valve 830 of the container 816 can be inserted into the valve retainer 830. Once in place, the container 816 is ready for use.

To use, a user twists the rotational stopping device 891 and unlocks the device 800. The user then moves the lid 844 from a closed position to an open position. This movement allows the valve block 860 to move in place and restrict the top portion 812 from activating the valve 830 of the container 816. Once opened, the user can insert an item 818 into the item holder 840 and then close the lid 844. A user then depresses the top portion 812 which in turn applies pressure to the container valve 830 and releases a pressurized gas. The gas enters the lower gas chamber 834 and, as pressure increase, the gas surrounds the item 818 thereby cooling/heating the item 818 to a desired temperature. If too much gas is applied, the gas can be directed downwards into the bottom portion 814 through openings 832 in a bottom portion of the chamber 834. This allows the gas to be controlled so that excess gas is not forced from any seams in the top portion 812 thereby causing injury to the user. Once a desired temperature is reached, the user can open the item holder 840 and remove the cooled item 818 for application, as needed.

The foregoing Detailed Description is to be understood as being in every respect illustrative, but not restrictive, and the scope of the disclosed technology disclosed herein is not to be determined from the Detailed Description, but rather from the claims as interpreted according to the full breadth permitted by the patent laws. It is to be understood that the implementations shown and described herein are only illustrative of the principles of the disclosed technology and that various modifications can be implemented without departing from the scope and spirit of the disclosed technology.

1. An apparatus comprising:
a top portion, the top portion includes (1) a valve receiver, the valve receiver capable of receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder capable of holding a removable item; and
a bottom portion, the bottom portion includes a base and sides, the sides extending upwards from the base, the sides forming an open cavity, the open cavity capable of receiving the container, wherein the bottom portion is movably coupled to the top portion.

2. The apparatus of claim 1 wherein the top portion further includes sides extending downwards, the sides of the top portion fitting within the open cavity of the bottom portion in a slidable configuration, so that, when downward pressure is applied to the top portion, the top portion is capable of actuating the valve of the container thereby releasing a pressurized medium into the exchange chamber.

3. The apparatus of claim 1 wherein the top portion further includes a lid movably coupled to the top portion.

4. The apparatus of claim 3 wherein the lid includes a portion of the exchange chamber.

5. The apparatus of claim 3 wherein the lid is connected to the top portion with a lever.

6. The apparatus of claim 5 wherein the lever includes an extender that actuates a valve block, the valve block preventing actuation of the valve when the lid is in an open position.

7. The apparatus of claim 1 wherein the exchange chamber includes at least one opening for releasing excess gas pressure from within the exchange chamber.

8. The apparatus of claim 1 wherein the removable item is a wand having a handle and a metallic head.

9. The apparatus of claim 7 wherein the item holder includes a seat for holding the metallic head and the handle.

10. The apparatus of claim 1 wherein the removable item is a tray for holding a composition.

11. The apparatus of claim 9 wherein the item holder includes a seat for holding the tray.

12. The apparatus of claim 1 wherein the top portion further includes a shell, the shell being fixably coupled to the top portion, the shell forming part of the exchange chamber.

13. The apparatus of claim 11 wherein the item holder pivots in a space created between the top portion and the shell.

14. The apparatus of claim 1 wherein the container is one of a gas container or an electric device.

15. The apparatus of claim 1 further including;
an outer casing and a twist-on cap.

16. An apparatus comprising:
a top portion, the top portion including (1) a valve receiver, the valve receiver receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder holding a composition packet; and
a bottom portion, the bottom portion including a base and sides, the sides extending upwards from the base, the sides forming an open cavity, the open cavity receiving the container, the bottom portion is movably coupled to the top portion.

17. The apparatus of claim 16 further including:
a lid; and
a lever connected to the lid, the lever including an extender that actuates a valve block, the valve block preventing actuation of the valve when the lid is in an open position.

18. The apparatus of claim 16 wherein the exchange chamber includes at least one opening for releasing excess gas pressure from within the exchange chamber.

19. An apparatus comprising:
a top portion, the top portion including (1) a valve receiver, the valve receiver receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder holding a wand having a handle and metallic head; and
a bottom portion, the bottom portion including a base and sides, the sides extending upwards from the base, the sides forming an open cavity, the open cavity receiving the container, the bottom portion is movably coupled to the top portion.

20. The apparatus of claim 19 wherein the exchange chamber includes at least one opening for releasing excess gas pressure from within the exchange chamber.

21. An apparatus comprising:
a top portion, the top portion includes (1) a valve receiver, the valve receiver capable of receiving a valve of a container, (2) an exchange chamber, (3) at least one aperture, the at least one aperture communicatively connecting the valve receiver to the exchange chamber, and (4) an item holder, the item holder being enclosed within the exchange chamber when the exchange chamber is in a closed position, the item holder capable of holding a removable item; and
a bottom portion, the bottom portion includes a base and an actuator, wherein the actuator is movably coupled to the base and is capable of actuating the valve of the container thereby releasing a pressurized medium into the exchange chamber.

22. The apparatus of claim 21 wherein the top portion and bottom portion are secured to each other with attachment threads.

23. The apparatus of claim 21 further comprising:
a recess, the recess allowing the item holder to pivot from an open position to a closed position within and out of the recess.

24. The apparatus of claim 21 wherein the actuator further includes a seat capable of receiving the container.

25. The apparatus of claim 24 wherein the seat is positioned slightly above a bottom wall of the actuator so as to create a space beneath the container.

26. The apparatus of claim 25 wherein the space creates an insulating distance between a bottom of the container and the bottom wall of the actuator.

27. An apparatus comprising:
a top portion, the top portion includes (1) a valve receiver, the valve receiver capable of receiving a valve of a container, (2) an exchange chamber, (3) at least one aperture, the at least one aperture communicatively connecting the valve receiver to the exchange chamber, (4) an item holder, the item holder being enclosed within the exchange chamber when the exchange chamber is in a closed position, the item holder capable of holding a removable item, and (5) a rotational locking device for locking the device when the exchange chamber is in a closed position.

28. The apparatus of claim 27 where the top portion is removably coupled to the container.
